(19) United States
(12) Patent Application Publication
(10) Pub. No.: US 2002/0143311 A1
(43) Pub. Date: Oct. 3, 2002

(54) Absorbent articles kit

(76) Inventor: Henri Brisebois, Lachenaie (CA)

Correspondence Address: AUDLEY A. CIAMPORCERO JR., JOHNSON & JOHNSON, ONE JOHNSON & JOHNSON PLAZA, NEW BRUNSWICK, NJ, 08933-7003, US

(21) Appl. No.: 09/822,878

(22) Filed: Mar. 30, 2001

(51) Int. Cl. A61F 13/15

(52) U.S. Cl. 604/385.01; 604/385.04; 604/379; 604/380; 206/440

(57) ABSTRACT

A kit of sanitary absorbent articles, including at least two sanitary absorbent articles stored in a container. The sanitary absorbent articles can be used separately or they can be assembled in a compound absorbent structure suitable for placement in a crotch portion of the undergarment of the wearer for absorbing menses. In a non-limiting example of implementation, one of the sanitary absorbent articles is a panty liner, the other sanitary absorbent article is a sanitary napkin without any flaps. The panty liner can be associated with the sanitary napkin in a transverse orientation such as to form flaps that can be folded about the longitudinal side edges of the crotch portion of the undergarment. Accordingly, if the user desires a sanitary napkin without flaps, she employs the sanitary napkin alone. If she desires a sanitary napkin with flaps, she combines the sanitary napkin with a panty liner to provide flaps to the sanitary napkin. Also, the user can employ a panty liner separately from a sanitary napkin, when the flow of bodily exudate is limited.

### FIELD OF THE INVENTION

The present invention relates to disposable articles used to absorb and retain bodily exudates such as sanitary napkins, panty liners and the like, and more particularly to a kit of absorbent articles including different categories of absorbent articles that can be combined with one another to provide structures offering particular features. In addition, the invention extends to absorbent articles that can be installed in different orientations in the crotch portion of the undergarment of the user.

### BACKGROUND OF THE INVENTION

Sanitary absorbent articles for the collection of menses and other bodily exudates can be categorized by the amount of fluid they can absorb. As an example, in the case of feminine sanitary protection there are sanitary napkins intended for medium to high menstrual flow and there are napkins commonly referred to as panty liners that are used to contain light or low menstrual flows. Panty liners are generally thinner and lack the absorptive capacity of the former category.

Sanitary absorbent articles can further be characterized by the presence or absence of flaps. The flap concept is extensively disclosed in the literature and sanitary napkins are available in the marketplace with flaps of various shapes and dimensions. Commonly, the flaps are provided with an adhesive means for attaching the flaps to the underside of a wearer's undergarment. When such flaps are in use they secure the napkin to the undergarment, making the napkin more stable. Flaps may comprise integral extensions of a material from which the article is formed, or alternatively, may comprise distinct pieces of material added to the article after its formation.

While sanitary absorbent articles having flaps are thought to perform better when compared to sanitary napkins without flaps, some users still prefer sanitary articles without flaps, while others who generally prefer a sanitary napkin with flaps, occasionally may not desire such features. For instance, flaps may not be required on days where the flow of exudate is light or when wearing a garment where flaps would be visible (such as a body suit). In this case, a panty liner or a sanitary napkin without flaps would be sufficient to meet a user's needs.

For the foregoing reasons, there exists a consumer need for a system whereby a sanitary napkin can be selectively provided with flaps to suit the specific needs of the user.

### SUMMARY OF THE INVENTION

In a first broad aspect, the invention provides a kit of sanitary absorbent articles, including:

a first sanitary absorbent article characterized by a total absorption capacity;

a second sanitary absorbent article characterized by a total absorption capacity, the total absorption capacity of the second sanitary absorbent article being less than the total absorption capacity of the first sanitary absorbent article;

the first sanitary absorbent article and the second sanitary absorbent article are capable of being used separately by a wearer for absorbing body exudate by placement in a crotch portion of an undergarment of the wearer;

the second sanitary absorbent article capable of being associated with the first sanitary absorbent article to form compound sanitary absorbent article having a pair of flaps capable of being folded about a crotch portion of the undergarment;

a container holding the first and the second sanitary absorbent articles.

In a specific non-limiting example of implementation under the first broad aspect of this invention, the first sanitary absorbent article is a sanitary napkin and the second sanitary absorbent article is a panty liner. The panty liner has an overall lesser absorption capacity than does the sanitary absorbent article. The panty liner and the sanitary napkin can be used separately if the user so desires, however, for users who prefer an absorbent article with flaps, the panty liner can be combined with the sanitary napkin to form an absorbent product with flaps. Optionally, the panty liner is provided with positioning adhesive on its garment-facing side. When associated with the sanitary napkin, the positioning adhesive serves the purpose of fastening the flaps formed by the panty liner to the outside of the undergarment (garment facing surface of the undergarment). When the panty liner is used separately from the sanitary napkin, the positioning adhesive serves the usual function of retaining the panty liner against the crotch portion of the undergarment of the wearer.

When the user desires an absorbent structure with flaps, the panty liner is mounted to the sanitary napkin such that in use the sanitary napkin overlies the panty liner. This is an advantageous mode of association of the panty liner and the sanitary napkin. However, the reverse arrangement can also be used without departing from the spirit of the invention, where the panty liner is placed over the sanitary napkin.

Prior to use, both the panty liner and the sanitary napkin are stored after their manufacture in a container, such as a cardboard box, a pouch of plastic material or any other suitable form of packaging. The packaging keeps the sanitary absorbent articles together, protects them and prevents contamination during shipping to the user. In the context of the invention, “container” implies a holding structure that provides a storage function and allows a user to access and remove the sanitary absorbent articles when desired. The container may hold only a single pair of sanitary absorbent articles, namely one sanitary napkin and one panty liner. Alternatively, the container may hold a multitude of sanitary absorbent articles, arranged according to different ratios. In one possibility, half of the sanitary absorbent articles are sanitary napkins while the other half are panty liners. In a different possibility, the ratio of sanitary napkins versus panty liners is different from 1:1, depending upon the specific application.

In a second broad aspect, the invention provides a sanitary absorbent article for placement in a crotch portion of an undergarment, the sanitary absorbent article comprising:

a main body including a body-facing side for placement toward a perineal region of the wearer and a garment-facing side opposite the body-facing side for placement toward the crotch portion of the undergarment;

a pair of opposite transverse end portions;

a pair of opposite longitudinal side portions, extending between the transverse end portions;

an imaginary transverse centerline;

an imaginary longitudinal centerline;

a pair of elongated preferential folding zones extending generally transversely to the longitudinal centerline, the elongated preferential folding zones being spaced from one another such that when the main body is placed transversely on the crotch portion of the undergarment, the elongated preferential folding zones register with respective longitudinal side edges of the crotch portion.

In a non-limiting example of implementation under the second broad aspect, the sanitary absorbent article is in the form of a panty liner. The folding zones are such that they permit the panty liner to be folded in the transverse direction around the longitudinal sides of the crotch portion of the undergarment.

In a third broad aspect, the invention provides a combination, comprising:

a first sanitary absorbent article;

a second sanitary absorbent article;

a container, the first and the second sanitary absorbent articles being placed in the container;

an item of information directing a user to associate the first and the second sanitary absorbent articles to form a compound sanitary absorbent structure that is suitable for placement in a crotch portion of an undergarment of the user for absorbing menstrual liquid.

In a non-limiting example of implementation under the third broad aspect of the invention, the first sanitary absorbent article is a sanitary napkin while the second sanitary absorbent article is a panty liner. The sanitary napkin and the panty liner can be associated with one another to form a compound sanitary absorbent structure that is suitable for placement in the crotch portion of the user's undergarment to absorb menses. Advantageously, the container is made of cardboard or it is under the form of a pouch of synthetic material, such as polymeric film, that provides suitable protection for the sanitary absorbent articles during storage and transportation. The item of information can take various forms. One possibility is to print the information, such as a notice, on the container, such that the user can discern or interpret the significance of the information prior to use of the sanitary absorbent articles. Another possibility is to print the notice on a leaflet and insert it in the container. The information contained in the item of information can be either in written form or graphic form, such as pictorial symbols to represent meaning, or a combination of both.

In another broad aspect, the invention provides a method for providing user-selective sanitary protection comprising the steps of:

providing a user with a container including at least one sanitary napkin and at least one panty liner, the sanitary napkin and the panty liner being capable of being used separately or assembled to one another to form a compound sanitary absorbent article;

providing the user with instructions on how to assemble the sanitary napkin and the panty liner into the compound sanitary absorbent article.

In yet another broad aspect, the invention provides a method for using two or more sanitary absorbent articles, each article containing a longitudinal axis, a body facing side and a garment facing side having one or more areas of attachment adhesive; the method comprising the steps of:

(a) positioning the second sanitary article transversely in a crotch portion of an undergarment of a wearer;

(b) positioning the first sanitary article in vertical registration with the second sanitary article such that the longitudinal axis of the first sanitary article is approximately perpendicular to the longitudinal axis of the second sanitary article;

(c) wherein at least one of the attachment adhesive areas of the second sanitary article secures the second sanitary article to the undergarment and wherein at least one of the attachment adhesive areas of the first sanitary article secures the first sanitary article to the second sanitary article.

### BRIEF DESCRIPTION OF THE DRAWINGS

The invention will now be described in greater detail with reference to the accompanying drawings, in which:

FIG. 1 is a top plan view of a panty liner in accordance with a non-limiting example of implementation of the invention;

FIG. 2 is a cross-sectional view of the panty liner taken along line 2-2 in FIG. 1;

FIG. 3 is a top plan view of a sanitary napkin in accordance with a non-limiting example of implementation of the invention;

FIG. 4 is a cross-sectional view of the sanitary napkin taken along Line 4-4 in FIG. 3;

FIG. 5 is a top plan view of the panty liner transversely placed beneath the sanitary napkin to form flaps for the sanitary napkin;

FIGS. 6 and 7 are variants illustrating different assembly configurations between the panty liner and the sanitary napkin;

FIG. 8 illustrates the combination panty liner and sanitary napkin placed on the crotch portion of the undergarment, before the transverse end portions of the panty liner have been folded to form flaps;

FIG. 9 is a general illustration of a kit of sanitary absorbent articles according to a non-limiting example of implementation of the invention; and

FIG. 10 is a bottom plan view of the panty liner illustrated in FIGS. 1 and 2, illustrating the pattern of positioning adhesive.

In the drawings, preferred embodiments of the invention are illustrated by way of example. It is to be expressly understood that the description and drawings are only for purposes of illustration and as an aid to understanding, and are not intended to be a definition of the limits of the invention.

### DETAILED DESCRIPTION OF THE INVENTION

In one aspect the present invention provides a kit of sanitary absorbent articles, which in a non-limiting example of implementation are a sanitary napkin and a panty liner.

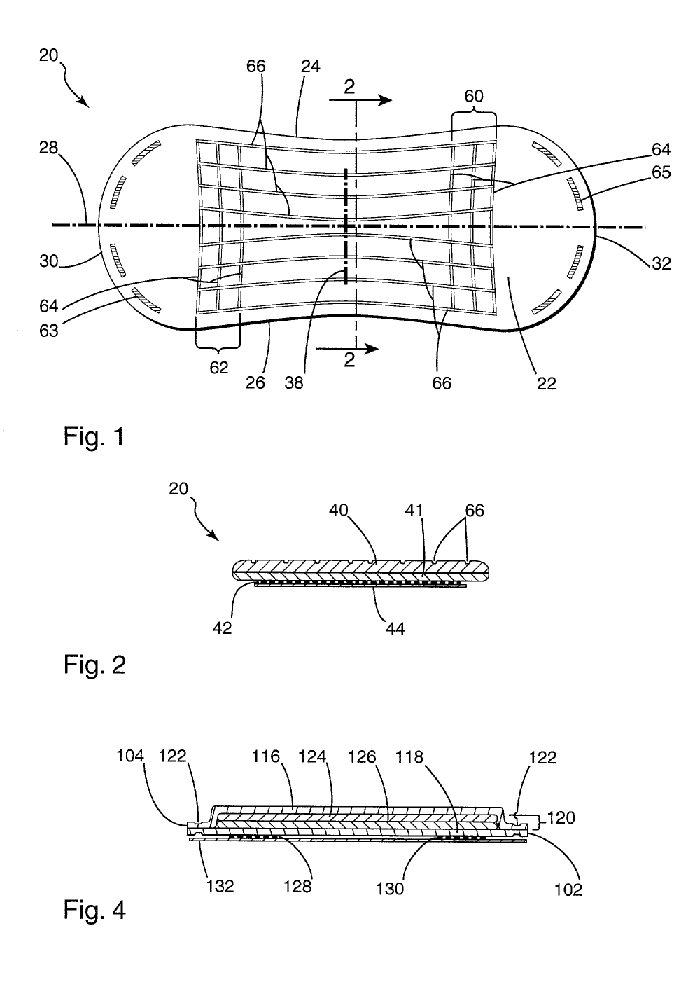

FIG. 1 illustrates one possible form of implementation of the panty liner, which is designated by the reference numeral 20. The panty liner comprises a main body 22. The main body 22 has two generally opposing longitudinal sides 24, 26 and an imaginary longitudinal centerline 28 running down the center of the panty liner 20, generally equidistant from the longitudinal sides 24, 26.

The main body 22 also has two generally opposing transverse sides 30, 32. The transverse sides are bi-convexly arcuate. An imaginary transverse centerline 38 runs across the panty liner 20, perpendicular to the longitudinal centerline 28.

In most instances, the longitudinal centerline 28 is an imaginary line that extends longitudinally along the main body 22, and it is equidistant from the longitudinal sides 24, 26. It will thus bisect the panty liner 20 into two generally mirror image halves. As the panty liner 20 is worn in the pudendal region, when the panty liner 20 is in use by a wearer, the longitudinal centerline 28 is generally parallel to, or most commonly, lies in, the sagittal plane of the wearer. Similarly, in most instances, the transverse centerline 38 is an imaginary line that extends transversely across the main body 22, and is typically, but not always, equidistant from the transverse sides 30, 32. The transverse centerline 38 is thus perpendicular to the longitudinal centerline 28.

With reference to FIG. 2, the panty liner 20 includes an absorbent layer 40 made of any suitable material, such as pulp fluff or non-woven fabric. The absorbent capacity of the layer 40 is limited because it is typical for the user to employ the panty liner only near the end of the menstrual period where the amount of body exudate discharged on the panty liner is small. Alternatively, the panty liner 20 may include other absorbent materials such as sphagnum moss, superabsorbents or the like. In use the top surface of the absorbent layer 40 faces the body of the wearer and it is referred in this specification as “body-facing side”. Immediately adjacent to the lower surface of the absorbent layer 40 (i.e., the surface that faces the garment of the wearer in use, referred to in this specification as “garment-facing side”), there is a barrier layer 41 with one or more adhesive zones 42 (described in greater detail later) for fastening the panty liner to the undergarment of the wearer.

In a non-limiting example of implementation, the barrier layer 41 is made of liquid-impervious film material so as to prevent liquid that is entrapped in the absorbent layer 40 from egressing the panty liner 20 and staining the wearer's undergarment. Most preferably, the barrier layer 41 is made of polymeric film, such as polyethylene or a polyethylene/ethylvinyl acetate (EVA), which are both inexpensive and readily available. The polymeric film is capable of fully blocking the passage of liquid or gas that may egress the absorbent layer 40. In a variant, breathable films may be used that allow passage of gases while blocking liquid. A suitable example is a combination polyethylene/ethylvinyl acetate (EVA) film sold by the Edison Plastics Company in the United-States under the commercial designation XP-1167B.

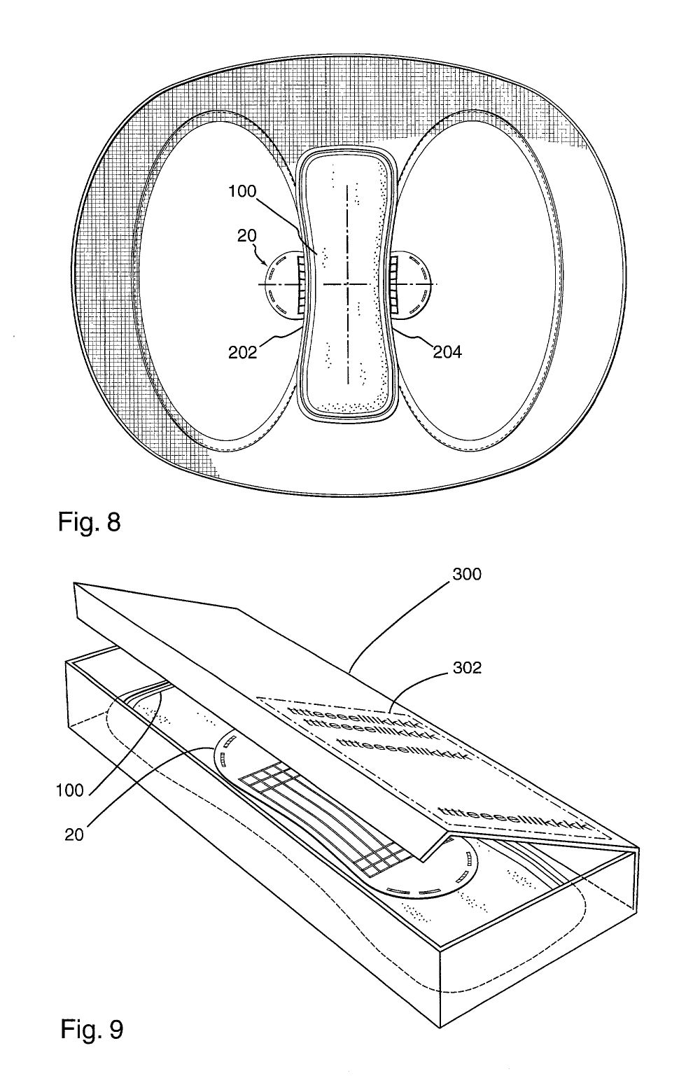

Referring back to FIG. 1, the main body 22 has two preferential folding zones 60, 62 which are disposed on either side of the transverse centerline 38. The folding zones 60, 62 extend transversely with relation to the longitudinal centerline 28 and have end portions that are adjacent to the respective longitudinal sides 24, 26. Each folding zone is a specific area of the main body 22 where the main body 22 is more likely to fold when subjected to bending stress than areas adjacent to that specific area. In one non-limiting example of implementation, each folding zone 60, 62 has at least one folding line 64 and preferably includes a series of folding lines 64 at which the main body 22 may fold. In the example shown in the drawings, each folding zone 60, 62 includes three arcuate folding lines 64. The folding lines 64 extend transversely of the centerline 28 of the panty liner and they are curved inwardly towards the center of the panty liner. The folding zones 60, 62 are spaced from another by a distance such that when the panty liner 20 is placed transversely on the crotch portion of an undergarment, the folding zones 60, 62 will register with the longitudinal (normally elasticized) sides of the crotch portion. This feature is best shown in FIG. 8. The folding zones 60, 62 are considered to register with the longitudinal sides of the crotch portion when during the folding of the panty liner 20 about the longitudinal side edges of the crotch portion the areas of the panty liner that bend or wrap around the longitudinal side edges of the crotch portion are contained at least partially in the folding zones 60, 62.

By providing each folding zone 60, 62 with a plurality of folding lines 64, a certain level of adaptability is achieved, allowing the actual fold to occur at the folding line 64 that best aligns with the respective longitudinal side of the crotch portion. This feature has been found advantageous particularly in instances where the panty liner 20 may be used on undergarments having different crotch widths. It should be expressly noted, however, that the present invention is not limited to this specific feature.

It should also be noted that in he preferred embodiment, the shape of the folding zones 60, 62 and the shape of the individual folding lines 64 are analogous to the shape of the longitudinal sides of the crotch portion of an undergarment, such as to facilitate folding. The panty liner also includes longitudinally extending folding lines 66 that are also arcuate. The folding lines reduce the longitudinal flexibility of the main body 22, allowing the panty liner 20 to better conform in use to the anatomy of the wearer. It should be expressly noted, however, that the present invention is not limited to having these specific features.

The folding lines 64, 66 are areas of the absorbent layer 40 where the absorbent material has been locally compressed to weaken it such as to create hinges. The embossing operation can be continuous, as depicted in the drawings, or discontinuous. Under the latter form of realization, each folding line is formed by a series of embossed areas, separated by non-embossed areas. The embossed areas may be dots or dashes or any other suitable geometric shape. The embossing operation to form the folding lines 64, 66 may be carried out by using embossing rolls or by any other suitable method, as known by those skilled in the art.

The main body 22 is provided near its transverse end portions 30, 32 with discontinuous embossing lines 63, 65 that are shaped to follow the contour of the transverse end portions 30, 32. The purpose of the discontinuous embossing lines 63, 65 is to reduce the possibility of menstrual or other liquid contained in the absorbent layer 40 from egressing the transverse end portions 30, 32. In particular, the embossed areas of the embossing lines 63, 65 are densified regions manifesting a lower average pore size than the average pore size of the non-embossed areas.

Accordingly, liquid that has migrated near the transverse end portions 30, 32 will have a tendency to accumulate in the embossed areas of the embossing lines 63, 65 and, in light of the fact that the embossed areas are discontinuous, the liquid will be trapped and will not have a tendency to wick away from the embossed areas.

It should be noted that the folding lines 64 and 66 also provide a certain level of liquid management function in addition to their purpose of facilitating the folding of the main body 22. The folding lines 64, 66 establish a network of liquid wicking channels that retain the liquid in the central area of the main body 22 and reduce the likelihood of liquid migrating toward the periphery of the main body 22.

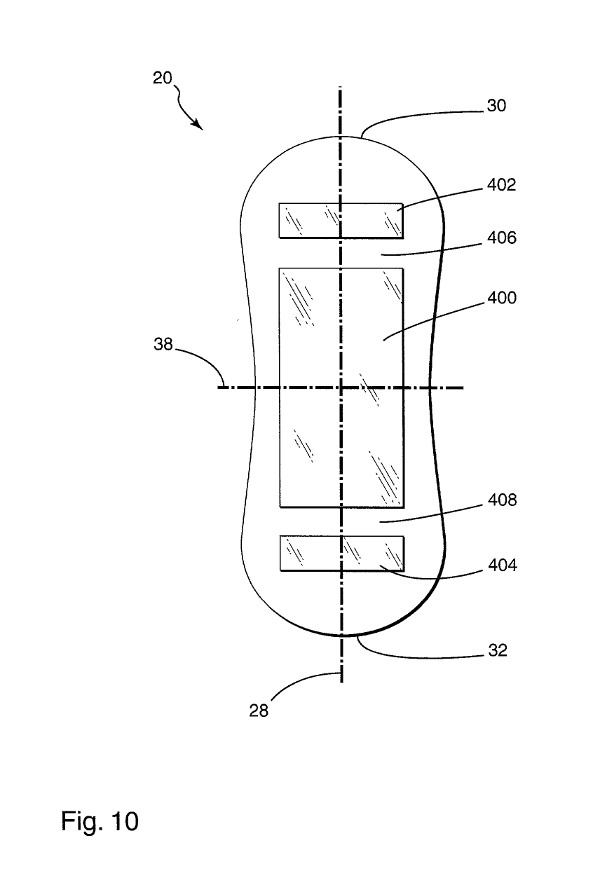

The pattern of application of the positioning adhesive 42 is illustrated in FIG. 10. The adhesive includes a centrally located main zone 400 and two smaller zones 402 and 404 spaced apart from the main zone 400 along the longitudinal centerline 28. The zones 400, 402 and 404 define between them two areas 406 and 408 that are devoid of adhesive and that register with the preferential folding zones 60, 62 respectively. The purpose of the adhesive-free zones 406 and 408 is to avoid establishing a bond with the longitudinal sides of the crotch portion of the undergarment when the panty liner 20 is transversely set on the crotch portion of the undergarment. In this fashion, if the longitudinal sides are elasticized, the elastics can perform their intended function (stretching and relaxing) with little impediment from the panty liner 20.

The adhesive zones 402 and 404 are located close enough to the transverse end portions 30 and 32 such that when the panty liner 20 is transversely set on the undergarment and the panty liner 20 folded at the preferential folding zones 60, 62 the adhesive zones 402 and 404 can bond with the garment facing surface of the undergarment.

The specific adhesive formulation can vary. It is within the reach of a person skilled in the art to select the proper adhesive formulation to suit the specific application. In one non-limiting example of implementation, the composition designated HL-1491 XZP, which is commercially available from H.B. Fuller Canada, Toronto, Ontario, Canada could be used. In general, the adhesive employed may be any one of a large number of pressure-sensitive adhesives that are commercially available, including water-based adhesives such as acrylate adhesives, e.g. vinyl acetate ethylhexylacrylate copolymer that may be combined with tackifiers. Alternatively, the adhesive may also comprise a double-faced tape.

A removable release paper 44 protects the adhesive coating 42. Prior to use, the release paper 44 is removed to expose the adhesive coating 42. A release paper under the form of a silicone coated wet-laid Kraft wood pulp could be used (available from Tekkote Corporation of Leonia, N.J., USA under the designation FRASER 30#/61629).

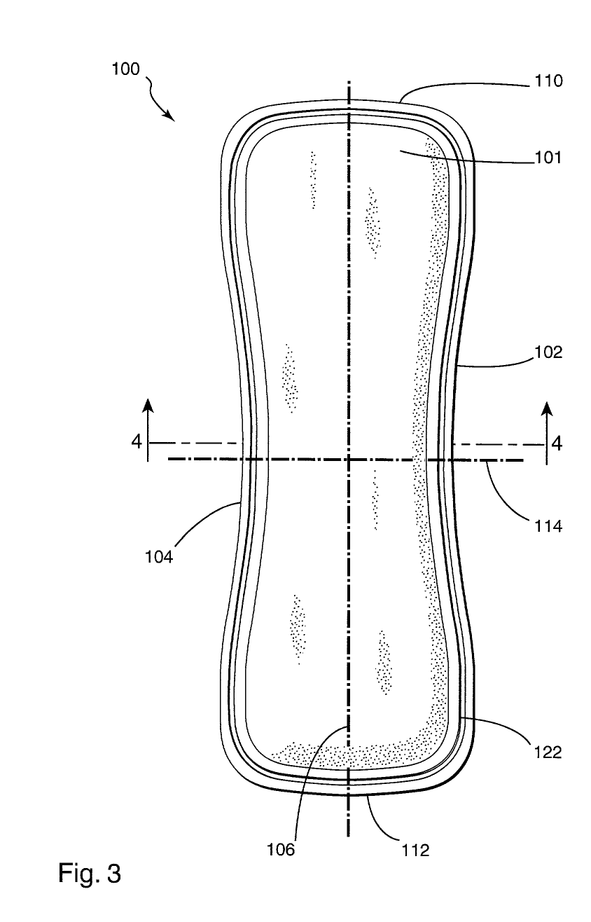

FIGS. 3 and 4 illustrate one possible form of implementation of the sanitary napkin that is designated by the reference numeral 100. As it is well known to those of ordinary skill in the art, the sanitary napkin 100 is used to absorb menstrual liquid, in particular during medium to high flow menstrual days of the user. To suit this purpose, the sanitary napkin 100 has a significantly higher absorption capacity than the panty liner 20. In other words, the sanitary napkin 100 can absorb and retain a significantly higher amount of liquid than the panty liner 20. To establish if an absorbent capacity differential exists between the two sanitary absorbent articles, the total capacity of the panty liner and the total capacity of the sanitary napkin are determined and then a comparison is made. The total capacity of a sanitary absorbent article is determined using the entire article minus any release paper. The article is weighed to the nearest 0.1 gram. The article is then submerged in a beaker of sterile saline (obtainable from the Baxter Travenol Company of Deerfield, Ill.), such that the article is totally submerged and is not bent or otherwise twisted or folded. The article is submerged for a period of 10 minutes. The article is then removed from the saline and suspended for two minutes in a vertical position to allow the saline to drain out of the article. The article is then placed body-facing surface down onto an absorbent blotter, such as the filter paper #631 available from the Filtration Science Corp., Eaton-Dikeman Division of Mount Holly Springs, Pa. A uniform 17.6 gram per square centimeter load is placed over the article to squeeze excess fluid out. The absorbent blotter is replaced every 30 seconds until the amount of fluid transferred to the absorbent blotter is less than 0.5 grams in a 30 second period. Next, the article is weighed to the nearest 0.1 gram and the dry weight of the article is subtracted. The difference in grams is the total capacity of the article.

The napkin 100 comprises a main body 101. The main body 101 has two generally opposing longitudinal sides 102, 104 and an imaginary longitudinal centerline 106 running down the center of the napkin 100, generally equidistant from the longitudinal sides 102, 104. The longitudinal sides 102, 104 are concavely arcuate.

The main body 101 also has two generally opposing transverse end portions 110, 112. The transverse end portions 110, 112 are convexly arcuate. An imaginary transverse centerline 114 runs across the napkin 100, perpendicular to the longitudinal centerline 106.

With reference to FIG. 4, the napkin 100 is a laminate structure and the main body 101 has an upper layer 116, which will face the body of a wearer when the napkin 100 is in use. This layer 116 is referred to in this specification as “cover layer”. The napkin 100 also includes a lower layer 118, designated as “barrier layer”, which will face the environment (i.e. away from the body of the wearer, and in almost all cases the wearer's undergarment) when the napkin 100 is in use. The sanitary napkin 100 also includes a sheet-like absorbent system 120 between the cover layer 116 and the barrier layer 118. The absorbent system 120 may be made of a single layer of absorbing material, but preferably it has a dual-layer construction, including an upper layer 124, called “transfer layer” and a lower layer 126, called “absorbent layer”. Under this form of implementation, the transfer layer 124 provides the means of receiving body fluid from the cover layer 116 and holding it until the absorbent layer 126 has an opportunity to absorb the fluid.

The cover layer 116 and the barrier layer 118 are sealed together along their peripheral edges to form a peripheral seal 122, containing the absorbent system 120. The seal may be made by means of adhesives, heat-bonding, ultrasonic bonding, radio frequency sealing, mechanical crimping and the like, or combinations thereof.

Each of these layers will be described in further detail below.

### Cover Layer

The purpose of the cover layer 116 is to provide an interface that would normally contact the body of the wearer when the sanitary napkin 100 is in use. The cover layer 116 is porous to liquids since its main function is to capture as quickly as possible a discharge of bodily exudate and transfer it to the absorbent system 120 underneath.

Under a non-limiting example of implementation, the cover layer 116 is formed from an apertured thermoplastic film. Such films are common in the art. An example is the co-extruded film described in U.S. Pat. No. 4,690,679, and marketed as RETICULON brand on sanitary napkins available from Johnson & Johnson Inc. of Montreal, Canada. Because of the high porosity of such films, they accomplish the function of quickly transferring body exudate to the inner layers (i.e. the absorbent system 120) of the napkin 100.

Under a different non-limiting example of implementation, the cover layer 116 is made of fibrous materials, such as non-woven fibrous materials. The cover layer 116 may be composed of only one type of fiber, such as polyester, or may be composed of bicomponent or conjugate fibers having a low melting point component and a high melting point component. Bicomponent fibers may be made up of a polyester core and a polyethylene sheath. The use of appropriate bicomponent materials results in a fusible non-woven fabric. Examples of such fusible fabrics are described in U.S. Pat. No. 4,555,430. Using a fusible fabric increases the ease with which the cover layer 116 may be mounted to the barrier layer 118 at the flange seal area 122.

The fibers may be selected from a variety of natural and synthetic materials such as nylon, polyester, rayon (in combination with other fibers), cotton acrylic fiber and the like and combinations thereof An example is the multi-denier cover layer described in the U.S. patent application Ser. No. 08/780,193 assigned to Johnson & Johnson. It will be evident to the person skilled in the art that a wide variety of other types of non-woven fabric materials can also be used.

### Transfer Layer

Adjacent to the cover layer 116 on its inner side and bonded thereto is the optional fluid transfer layer 124, that may form part of the absorbent system 120.

The transfer layer 124 is, preferably, more dense than and has a larger proportion of smaller pores than the cover layer 116. These attributes allow the transfer layer 124 to contain body fluid and hold it away from the outer side of the cover layer 116, thereby preventing the fluid from re-wetting the cover layer 116 and its surface. However, the transfer layer 124 is, preferably, not so dense as to prevent the passage of the fluid through the transfer layer 124 into the absorbent layer 126 therebelow.

The transfer layer 124 may be composed of fibrous materials, such as wood pulp, polyester, rayon, flexible foam, or the like, or combinations thereof The transfer layer 124 may also comprise thermoplastic fibers for the purpose of stabilizing the layer and maintaining its structural integrity. The transfer layer 124 may be treated with surfactant on one or both sides in order to increase its wettability, although generally the transfer layer 124 is relatively hydrophilic and may not require treatment. The transfer layer 124 is preferably bonded on both sides to the adjacent layers, i.e. the cover layer 116 and the absorbent layer 126. An example is the material sold by Merfin in the United-States under the commercial designation VICELL 6002.

### Absorbent Layer

Immediately adjacent to and bonded to the transfer layer 124 is the absorbent layer 126 that forms part of the absorbent system 120. The absorbent system 120 may comprise only the absorbent layer 126 or it may comprise a plurality of layers, such as the absorbent layer 126 in combination with the transfer layer 124 or any other additional layer. This is to say that the transfer layer 124 is not an essential component of the present invention.

The absorbent layer 126 is preferably a highly dense layer having a fine porosity. It has a large liquid holding capacity. In a specific non-limiting example of implementation of the present invention, the absorbent layer 126 comprises pulp fluff material and may optionally include other absorbent materials or non-absorbent materials which aid in stabilizing the absorbent structure such as conjugate fibers, fusible fibers, binders, sphagnum moss, superabsorbent, and the like and combinations thereof.

In an alternative embodiment the absorbent layer 126 comprises sphagnum moss and may optionally include other absorbent materials or non-absorbent materials which aid in stabilizing the absorbent structure such as conjugate fibers, fusible fibers, binders, superabsorbent, and the like and combinations thereof.

### Barrier Layer

Underlying the absorbent system 120 is a barrier layer 118 that may be identical to the barrier layer 41 of the panty liner 20 described earlier.

### Adhesives

Referring to FIG. 4, in order to secure the napkin 100 to the undergarment of a wearer, the barrier layer 118 is provided with a plurality of areas of adhesive material 128, 130 on the environmental (i.e. undergarment) facing surface thereof Specifically, the areas of adhesive material are in the form of two strips extending along the longitudinal centerline 106. A single release paper 132 covers the adhesive areas 128, 130. A suitable adhesive is the composition designated HL-1491 XZP commercially available from H.B. Fuller Canada, Toronto, Ontario, Canada. The release papers are of conventional construction (silicone coated wet-laid Kraft wood pulp) and suitable papers are available from Tekkote Corporation (Leonia, N.J., USA), and bear the designation FRASER 30#/61629.

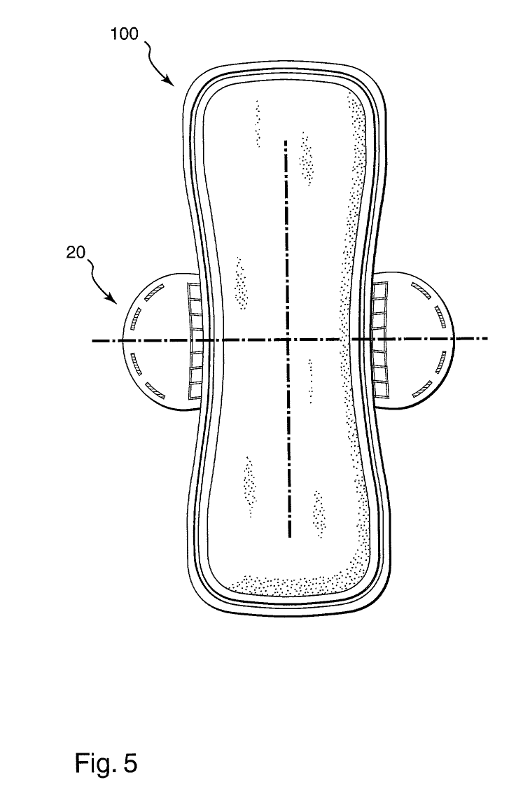

The manner in which the panty liner 20 and the sanitary napkin 100 are used will now be described with particular reference to FIG. 5 and FIG. 8. If desired, the two products can be used separately. In such case, the sanitary napkin 100 is a napkin without flaps. When the products are to be used separately, the user removes the release paper 44, 132 and places the selected product longitudinally in the crotch portion of the undergarment.

The sanitary napkin 100 shown in the drawings is flapless, in other words free of flaps projecting from its longitudinal sides 102, 104. If one desires to convert the sanitary napkin 100 to a product with flaps, then the two products can be associated to form a compound absorbent structure. The first step is to place the panty liner 20 in the crotch portion of the undergarment (after removal of the release paper 44), but in a transverse direction. The panty liner 20 is placed such as to register the preferential folding zones 60, 62 with the longitudinal side edges 202, 204 of the undergarment. The sanitary napkin 100 is then placed over the panty liner 20, in the usual fashion, in the crotch portion of the undergarment. The transverse ends 30, 32 of the panty liner 20 protruding from the undergarment are folded around the longitudinal side edges 202, 204 and secured to the outer surface of the crotch area of the undergarment with the adhesive 42, in the same way flaps of a sanitary napkin would be secured.

The ends of the panty liner 20 may or may not overlap each other, depending upon the width of the crotch portion of the undergarment. The folding of the panty liner 20 about the edges of the crotch portion of the undergarment is facilitated by the transverse folding zones 60, 62 that register with the longitudinal side edges of the undergarment.

The adhesive 128, 130 of the sanitary napkin 100 secures the sanitary napkin 100 to the cover layer of the panty liner 20 so that the two products are securely fastened to one another.

In an alternative embodiment, not shown in the drawings, the sanitary napkin 100 can be provided with its own flaps, and the association with the panty liner 20 may be used to alter the configuration of the flaps such as to yield longer flaps (measured along the transverse centerline 38) or wider flaps (measured along the longitudinal centerline 28). The characteristics of the flaps resulting from the association between the panty liner 20 and the sanitary napkin 100 will depend to a large degree upon the geometrical configuration of the panty liner 20, namely its length and width characteristics.

In yet another possible embodiment, more than one panty liner 20 can be associated with the sanitary napkin 100. In particular, two or more panty liners 20 can be associated with the sanitary napkin 100 such that each panty liner 20 extends transversely with relation to the sanitary napkin 100, and the panty liners are set at different locations along the longitudinal centerline of the sanitary napkin 100.

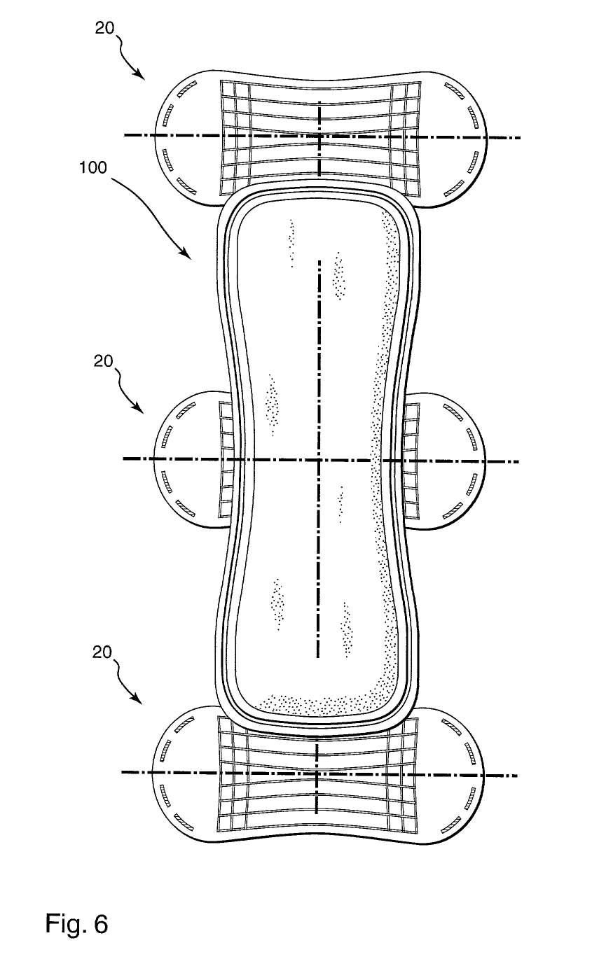

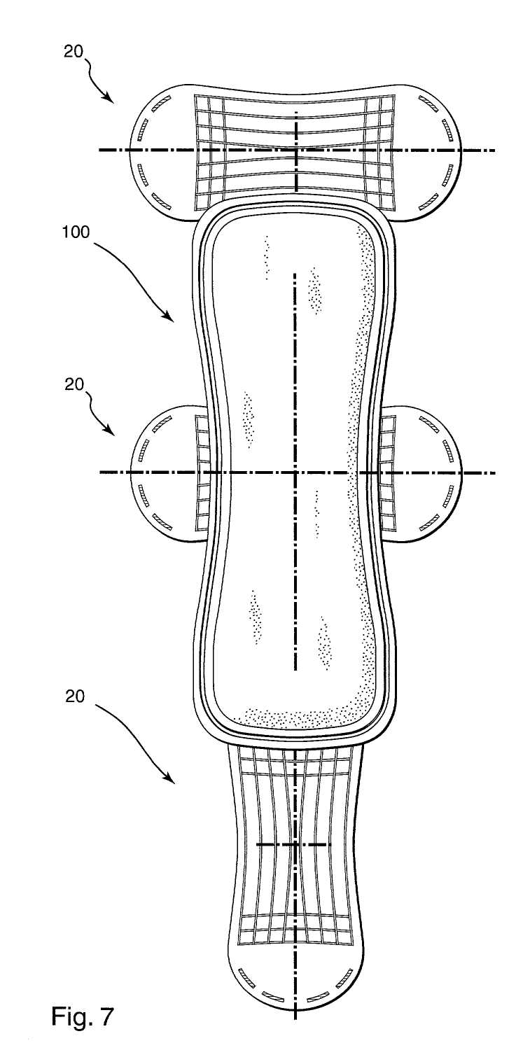

FIGS. 6 and 7 illustrate other possible combinations between the panty liner(s) 20 and the sanitary napkin 100. All these combinations are realized by using the adhesive areas 128, 130 of the sanitary napkin 100 and the adhesive(s) 42 of the panty liner(s) 20 as bonding agents between the sanitary napkin 100 and the panty liner(s) 20. As shown in FIG. 6, three panty liners 20 are positioned transversely relative to the sanitary napkin 100. One panty liner 20 is positioned beneath the center portion of the sanitary napkin 100, to provide flaps, and the other two panty liners 20 are placed beneath each transverse end of the sanitary napkin 100. FIG. 7 illustrates a variation of FIG. 6 wherein one of the two panty liners 20 at the transverse ends of the sanitary napkin 100 is placed in the same direction as the sanitary napkin 100.

Prior to use, the panty liner 20 and the sanitary napkin 100 are stored in a container 300 shown in FIG. 9. The example of FIG. 9 shows the container 300 in the form of a cardboard box, however, other forms of packaging can be used without departing from the spirit of the invention, such as a flexible pouch of synthetic material or any other suitable structure that physically combines both absorbent products such that they can be readily accessed by the user and employed either separately or in combination. In a preferred arrangement, the container 300 houses a plurality of panty liners 20 and a plurality of sanitary napkins 100 but a container 300 containing a single sanitary napkin 100 and a single panty liner 20 is within the scope of this invention. The ratio of panty liners 20 to sanitary napkins 100 could be 1:1 or a different ratio (i.e., 2:1 or 3:1). To provide instructions on how to associate the panty liner and the sanitary napkin, the container 300 includes an item of information 302. This item of information 302 can be in written form or as iconic symbols or a combination of both. In general, the item of information 302 is realized on a suitable substrate and it is physically associated with the container 300, such as by printing it on the container 300 (as shown in FIG. 9 where the container 300 forms the substrate), or placing a leaflet or the like in the container 300. Alternatively, the item of information can be formed on the release paper 44 or the release paper 132 or both.

The panty liner 20 and the sanitary napkin 100 can be stored in the container 300 as depicted in the drawings, in particular the panty liner 20 and the sanitary napkin 100 are deposited in the container 300 without any other form of individual packaging. It is also possible without departing from the spirit of invention to individually package the panty liner 20 and the sanitary napkin 100 and then place them in the container 300. The individual packaging can be in the form of a pouch or bag of synthetic material that encloses completely or partially the panty liner 20 or the sanitary napkin 100.

Although various embodiments have been illustrated, this was for the purpose of describing, but not limiting, the invention. Various modifications will become apparent to those skilled in the art and are within the scope of this invention, which is defined more particularly by the attached claims.

1. A kit of sanitary absorbent articles, comprising:
a first sanitary absorbent article characterized by a total absorption capacity;
a second sanitary absorbent article characterized by a total absorption capacity, the total absorption capacity of said second sanitary absorbent article being less than the total absorption capacity of said first sanitary absorbent article;
said first sanitary absorbent article and said second sanitary absorbent article are capable of being used separately by a wearer for absorbing body exudate by placement in a crotch portion of an undergarment of the wearer;
said second sanitary absorbent article capable of being associated with said first sanitary absorbent article to form a compound sanitary absorbent article having a pair of flaps capable of being folded about a crotch portion of the undergarment;
a container holding said first and said second sanitary absorbent articles.

2. A kit as defined in claim 1, wherein said first sanitary absorbent article is a sanitary napkin.

3. A kit as defined in claim 2, wherein said second sanitary absorbent article is a panty liner.

4. A kit as defined in claim 1, wherein said second sanitary absorbent article is capable of being associated with said first sanitary absorbent article such that said second sanitary absorbent article extends transversely with relation to said first sanitary absorbent article to form the pair of flaps.

5. A kit as defmed in claim 4, wherein said first sanitary absorbent article includes an adhesive capable of bonding said first sanitary absorbent article to said second sanitary absorbent article.

6. A kit as defined in claim 4, wherein said second sanitary absorbent article includes a main body, said main body having:
a body-facing side for placement toward a perineal region of the wearer;
a garment-facing side opposite said body-facing side for placement toward the crotch portion of the undergarment, the crotch portion being characterized by longitudinal side edges;
a pair of opposite transverse end portions and a pair of opposite longitudinal side portions, extending between said transverse end portions;
an imaginary transverse centerline;
an imaginary longitudinal centerline;
a pair of elongated preferential folding zones extending generally transversely to said longitudinal centerline, said elongated preferential folding zones being spaced from one another such that when said main body is placed transversely on the crotch portion of the undergarment, said elongated preferential folding zones register with respective longitudinal side edges of the crotch portion.

7. A kit as defined in claim 6, wherein each preferential folding zone includes a preferential folding line extending generally transversely with respect to said longitudinal centerline, said preferential folding line.

8. A kit as defined in claim 7, wherein each preferential folding zone includes a plurality of preferential folding lines.

9. A kit as defined in claim 7, wherein said preferential folding line is an embossed line realized on said main body.

10. A kit as defmed in claim 9, wherein said embossed line is arcuate, curving toward a center portion of said main body.

11. A kit as defmed in claim 10, wherein said embossed line is realized by continuous embossing.

12. A kit as defined in claim 6, wherein said garment facing side includes two zones having positioning adhesive, said zones capable of establishing an adhesive bond with a garment facing surface of the crotch portion of the undergarment when the flaps formed by said second sanitary absorbent article are folded about the longitudinal side edges of the undergarment.

13. A kit as defined in claim 12, wherein said garment facing side includes areas that are substantially free of positioning adhesive and that register with said preferential folding zones.

14. A sanitary absorbent article for placement in a crotch portion of an undergarment having longitudinal side edges, said sanitary absorbent article comprising:
a main body including a body-facing side for placement toward a perineal region of the wearer and a garment-facing side opposite said body-facing side for placement toward the crotch portion of the undergarment;
a pair of opposite transverse end portions;
a pair of opposite longitudinal side portions, extending between said transverse end portions;
an imaginary transverse centerline;
an imaginary longitudinal centerline;
a pair of elongated preferential folding zones extending generally transversely to said longitudinal centerline, said elongated preferential folding zones being spaced from one another such that when said main body is placed transversely on the crotch portion of the undergarment, said elongated preferential folding zones register with respective longitudinal side edges of the crotch portion.

15. A sanitary absorbent article as defmed in claim 14, wherein each preferential folding zone includes a preferential folding line extending generally transversely with respect to said longitudinal centerline.

16. A sanitary absorbent article as defined in claim 14, wherein each preferential folding zone includes a plurality of preferential folding lines.

17. A sanitary absorbent article as defined in claim 15, wherein said preferential folding line is an embossed line realized on said main body.

18. A sanitary absorbent article as defined in claim 17, wherein said embossed line is arcuate, curving toward a center portion of said main body.

19. A sanitary absorbent article as defined in claim 18, wherein said embossed line is realized by continuous embossing.

20. A sanitary absorbent article as defined in claim 18, wherein said main body includes an embossed line extending along each transverse end portion, that is discontinuous and includes areas of embossing separated by non-embossed areas.

21. A sanitary absorbent article as defined in claim 14, wherein said garment facing side includes two zones having positioning adhesive, the zones having positioning adhesive capable of establishing an adhesive bond with a garment facing surface of the crotch portion of the undergarment when the transverse end portions of said main body are folded about the longitudinal side edges of the undergarment.

22. A sanitary absorbent article as defined in claim 21, wherein said garment facing side includes areas that are substantially free of positioning adhesive and that register with said preferential folding zones.

23. A sanitary absorbent article as defined in claim 22, wherein said sanitary absorbent article is a panty liner.

24. A combination, comprising:
a first sanitary absorbent article;
a second sanitary absorbent article;
a container, said first and said second sanitary absorbent articles being placed in said container;
an item of information directing a user to associate said first and said second sanitary absorbent articles to form a compound sanitary absorbent structure that is suitable for placement in a crotch portion of an undergarment of the user for absorbing menstrual liquid.

25. A combination as defined in claim 24, wherein said first sanitary absorbent article is flapless.

26. A combination as defined in claim 25, wherein said second sanitary absorbent article is flapless.

27. A combination as defined in claim 24, wherein said item of information is realized on said container.

28. A combination as defined in claim 27, wherein said item of information includes a written notice.

29. A combination as defmed in claim 27, wherein said item of information includes pictorial symbols.

30. A combination as defined in claim 24, wherein said item of information is realized on a substrate stored in said container.

31. A method for providing user-selective sanitary protection comprising the steps of:
providing a user with a container including at least one sanitary napkin and at least one panty liner, the sanitary napkin and the panty liner being capable of being used separately or assembled to one another to form a compound sanitary absorbent article;
providing the user with instructions on how to assemble the sanitary napkin and the panty liner into the compound sanitary absorbent article.

32. A method of using two or more sanitary absorbent articles by a female wearer, wherein said sanitary absorbent articles comprise a first sanitary article and a second sanitary article and wherein each sanitary article contains a longitudinal axis, a body facing side and a garment facing side having one or more areas of attachment adhesive; said method comprising the steps of:
(a) positioning the second sanitary article transversely in a crotch portion of an undergarment of a wearer;
(b) positioning the first sanitary article in vertical registration with the second sanitary article such that the longitudinal axis of the first sanitary article is approximately perpendicular to the longitudinal axis of the second sanitary article;
(c) wherein at least one of said attachment adhesive areas of the second sanitary article secures the second sanitary article to the undergarment and wherein at least one of said attachment adhesive areas of the first sanitary article secures the first sanitary article to the second sanitary article.

33. The method of claim 32 wherein step (a) is performed before step (b).

34. The method of claim 32 wherein step (b) is performed before step (a).

35. The method of claim 32 wherein said undergarment has a body facing side and a garment facing side and wherein at least one of said attachment adhesive areas of the second sanitary article attaches to the garment facing side of the undergarment.
